(19) United States
(12) Patent Application Publication
(10) Pub. No.: US 2009/0198136 A1
(43) Pub. Date: Aug. 6, 2009

(54) ULTRASONIC DIAGNOSTIC APPARATUS

(76) Inventors: Koji Yanagihara, Tokyo (JP); Akimasa Asai, Tokyo (JP); Kazuhiko Hayakawa, Tokyo (JP); Hiroyuki Hatakenaka, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/363,298

(22) Filed: Jan. 30, 2009

(30) Foreign Application Priority Data
Jan. 31, 2008 (JP) 2008-020159
May 27, 2008 (JP) 2008-137563

(51) Int. Cl. A61B 8/00

(52) U.S. Cl. 600/443

(57) ABSTRACT

An ultrasonic diagnostic apparatus includes an image display device that displays an ultrasonic image, and a processing device that controls to generate an ultrasonic image on the basis of an echo signal obtained through the transmission of an ultrasonic wave and to display the ultrasonic image on the image display device. The image display device is mounted separate from the processing device, and attached at a ceiling or a wall of a room, to which the ultrasonic diagnostic apparatus is installed, through an arm, and the arm has a movable portion for changing the position of the image display device.

### CROSS REFERENCE TO RELATED APPLICATIONS

This application claims the benefit of Japanese Patent Application No. 2008-020159 filed Jan. 31, 2008, and Japanese Patent Application No. 2008-137563 filed May 27, 2008, each of which is hereby incorporated by reference in its entirety.

### BACKGROUND OF THE INVENTION

The embodiments described herein relate to an ultrasonic diagnostic apparatus that generates an ultrasonic image on the basis of an echo signal obtained through a transmission of an ultrasonic wave.

A conventional ultrasonic diagnostic apparatus is configured to include an operation device by which an operator inputs an instruction, an image display device that displays an ultrasonic image, and a processor that controls to generate and display ultrasonic image data on the basis of an echo signal obtained through the transmission of an ultrasonic wave. The operation device, the image display device, and the processor are integrally configured so as to be movable (see, for example, Japanese Unexamined Patent Publication No. 2003-339708).

The conventional ultrasonic apparatus is placed at the side where the operator is present with respect to a bed on which a patient lies, in order to put the operation device at the position accessible by the operator. Therefore, the operator has to place a probe to the subject, who lies down on the bed, for scan, while looking at the ultrasonic image displayed onto the image display device in the vicinity of the ultrasonic diagnostic apparatus at the bedside, which forces the operator to make a scan in a forced posture. Depending upon a region to be diagnosed, the subject may sit on the bed, and the operator may make a scan as bending down. In this case, the operator has to make a scan as looking up the image display device, so that the operator is forced to take an unnatural posture.

Since the operation device, the image display device and the processor are integrally formed, the space where the operator is present is greatly occupied when the large-sized conventional ultrasonic diagnostic apparatus is put at the side where the operator is present across the bed.

### BRIEF DESCRIPTION OF THE INVENTION

Embodiments of the invention provide an ultrasonic diagnostic apparatus by which an operator can make a scan in a natural posture.

Moreover, embodiments of the invention provide an ultrasonic diagnostic apparatus by which an operator can make a scan in a natural posture, and to effectively utilize the space of the room in which the ultrasonic diagnostic apparatus is installed.

According to one aspect, an ultrasonic diagnostic apparatus that includes an image display device that displays an ultrasonic image, and a processing device that controls to generate an ultrasonic image on the basis of an echo signal obtained through the transmission of an ultrasonic wave and to display the ultrasonic image on the image display device, wherein the image display device is mounted separate from the processing device, and attached at a ceiling or a wall of a room to which the ultrasonic diagnostic apparatus is installed, through an arm, and the arm has a movable portion for changing the position of the image display device.

In a second aspect, and according to the first aspect, the arm is configured to include plural arm members, and the movable portion is composed of a connection portion to which each of the arm members is pivotally connected.

In a third aspect, and according to the first and the second aspects, the movable portion is configured by the structure that the arm is extensible.

In a fourth aspect, and according to the first to the third aspects, the arm is mounted to a ceiling or a wall of a room, to which the ultrasonic diagnostic apparatus is installed, so as to be movable.

In a fifth aspect, and according to the first to the fourth aspects, the image display device and the processing device have a wireless communication unit for achieving a mutual wireless communication.

In a sixth aspect, and according to the first to the fifth aspects, the ultrasonic diagnostic apparatus also includes an operation device by which an operator inputs an instruction, wherein the processing device is provided separate from the operation device, and is mounted to a wall of the room to which the ultrasonic diagnostic apparatus is installed, or to the vicinity of the wall of the room.

In a seventh aspect, and according to the sixth aspect, the operation device is placed onto a movable table with casters.

In an eighth aspect, and according to the sixth aspect or the seventh aspect, the image display device and the processing device have a wireless communication unit for achieving a mutual wireless communication.

In a ninth aspect, and according to the first to the eighth aspects, a probe for transmitting and receiving the ultrasonic wave is connected to the processing device, the ultrasonic diagnostic apparatus including a cable storing unit that stores a cable for connecting a probe body of the probe with the processing device.

In a tenth aspect, and according to the ninth aspect, the cable storing unit has a cable storing casing having a storing space of the cable formed therein.

In an eleventh aspect, and according to the tenth aspect, the cable storing casing is formed with an opening at the position approximately 120.0 centimeters (cm) or more high from the floor of the room, and the cable is stored into the cable storing space from the opening.

In a twelfth aspect, and according to the tenth aspect or the eleventh aspect, wherein the processing device is integrally mounted above the cable storing casing in which the thickness of the portion having the height of approximately 70.0 cm or less from the floor of the room is approximately 15.0 cm or less.

In a thirteenth aspect, and according to the first to the twelfth aspects, the processing device has a portion that is approximately 70.0 cm or less high from the floor of the room, and the thickness of this portion is approximately 15.0 cm or less.

### ADVANTAGEOUS EFFECTS OF INVENTION

According to the first aspect, the image display device is mounted separate from the processing device, and attached at a ceiling or a wall of a room, to which the ultrasonic diagnostic apparatus is installed, through an arm having a movable portion for changing the position of the image display device. Therefore, the operator can place the image display device at the position where the operator can make a scan with a natural posture according to the posture of the operator upon the diagnosis.

According to the second aspect, each of the arm members is pivoted about the connection portion, whereby the operator can place the image display device at the position where the operator can make a scan with a natural posture.

According to the third aspect, the arm is extended and contracted, whereby the operator can place the image display device at the position where the operator can make a scan with a natural posture.

According to the fourth aspect, the arm is mounted to the ceiling or the wall of the room so as to be movable, whereby the operator can place the image display device at the position where the operator can make a scan with a natural posture. Further, when the operator does not use the apparatus, the operator can move the apparatus to the place in the room where the apparatus does not become obstructive.

According to the fifth aspect, the cable for connecting the image display device and the processing device can be eliminated, whereby it can be prevented that the movement of the image display device is hindered by the cable. Further, this configuration prevents the cable from being obstructive.

According to the sixth aspect, like the first aspect, the operator can make a scan with a natural posture. In addition, the operation device, which should be placed at the side where the operator is present across the bed on which the patient lies down, and the processing device are provided separately, and the processing device is mounted to the wall of the room or in the vicinity of the wall of the room, whereby the processing device can be arranged at the wall side while the operation device can be arranged at the side, which is opposite to the wall side and where the operator is present, across the bed. By virtue of this configuration, the space where the operator is present becomes wider than the conventional case because the processing device is not placed in this space, so that the space of the room can effectively be utilized.

According to the seventh aspect, the position of the operation device can freely be changed by moving the movable table.

According to the eighth aspect, the cable for connecting the processing device and the operation device can be eliminated, whereby the operation device can freely be moved. Further, this configuration prevents the cable from being obstructive.

According to the ninth aspect, the cable can be stored in the cable storing unit, which prevents the cable from being obstructive.

According to the tenth aspect, the cable can be stored in the cable storing casing, which prevents the cable from being obstructive.

According to the eleventh aspect, the cable storing casing is formed with an opening at the position 120 cm or more high from the floor of the room to which the ultrasonic diagnostic apparatus is installed, whereby the cable is inserted and removed at the position sufficiently higher than the patient lying down on the bed in the room. Accordingly, this configuration can prevent the patient from having an uncomfortable feeling by the cable.

According to the twelfth aspect, the height of a normal bed is less than 70 cm, and the processing device is integrally mounted above the cable storing casing in which the thickness of the portion having the height of 70 cm or less from the floor of the room is 15 cm or less. Therefore, the gap between the bed and the wall can be set to, for example, 16 cm, with the result that the bed can be made as closer to the wall as possible. Thus, the space where the operator is present becomes much wider than the conventional case, so that the space of the room, to which the ultrasonic diagnostic apparatus is installed, can effectively be utilized.

According to the thirteenth aspect, the thickness of the portion having the height of 70 cm or less from the floor of the room is 15 cm or less, whereby the gap between the bed and the wall can be set to, for example, 16 cm, with the result that the bed can be made as closer to the wall as possible, like the eleventh aspect. Thus, the space where the operator is present becomes much wider than the conventional case, so that the space of the room, to which the ultrasonic diagnostic apparatus is installed, can effectively be utilized.

Further objects and advantages of the present invention will be apparent from the following description of the preferred embodiments of the invention as illustrated in the accompanying drawings.

### BRIEF DESCRIPTION OF THE DRAWINGS

FIG. 1 is a partially cutout side view showing an exemplary ultrasonic diagnostic apparatus according to a first embodiment.

FIG. 2 is a front view of the ultrasonic diagnostic apparatus shown in FIG. 1.

FIG. 3 is a plan view of the ultrasonic diagnostic apparatus shown in FIG. 1.

FIG. 4 is a partially cutout enlarged side view showing the vicinity of an opening of the ultrasonic diagnostic apparatus shown in FIG. 1.

FIG. 5 is an enlarged front view showing the vicinity of an opening of the ultrasonic diagnostic apparatus shown in FIG. 1.

FIG. 6 is an enlarged side view showing the vicinity of a movable pulley when a cover attached to the cable storing casing is slid downward to expose the inside of the cable storing casing.

FIG. 7 is an enlarged side view of the vicinity of a movable pulley lock showing the state in which the movable pulley lock is released.

FIG. 8 is an enlarged side view of the vicinity of a movable pulley lock showing the state in which the movable pulley lock is operated.

FIG. 9 is an enlarged side view of the vicinity of the movable pulley showing the state in which the cable lock is released.

FIG. 10 is a partially cutout side view showing an exemplary ultrasonic diagnostic apparatus according to a second embodiment.

FIG. 11 is a front view showing the ultrasonic diagnostic apparatus shown in FIG. 10.

FIG. 12 is a front view showing an exemplary ultrasonic diagnostic apparatus according to a third embodiment.

FIG. 13 is a front view showing an exemplary ultrasonic diagnostic apparatus according to a fourth embodiment.

FIG. 14 is an upward view of a second horizontal slide guide according to the fourth embodiment.

FIG. 15 is a front view showing an ultrasonic diagnostic apparatus according to the fifth embodiment.

FIG. 16 is a front view showing an exemplary ultrasonic diagnostic apparatus according to a sixth embodiment.

### DETAILED DESCRIPTION OF THE INVENTION

Embodiments of the invention will be described in detail below with reference to the drawings.

### First Embodiment

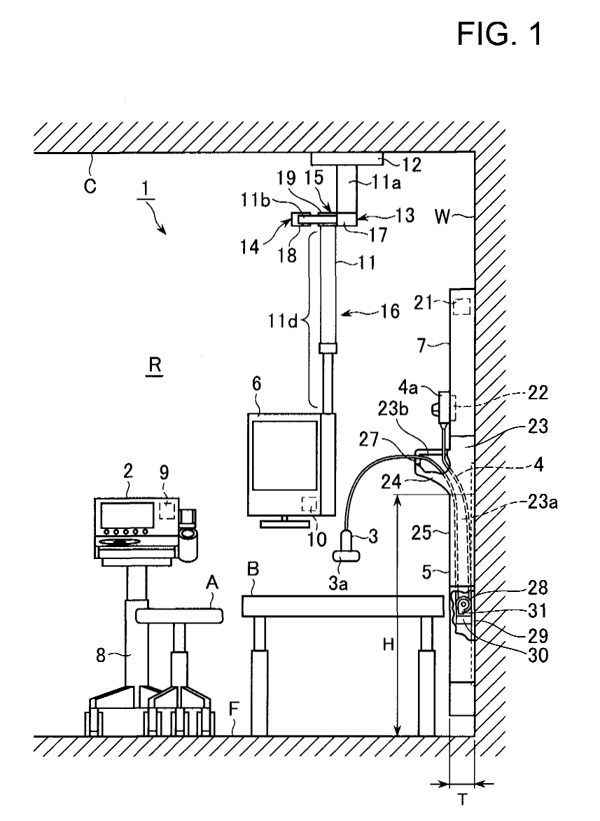

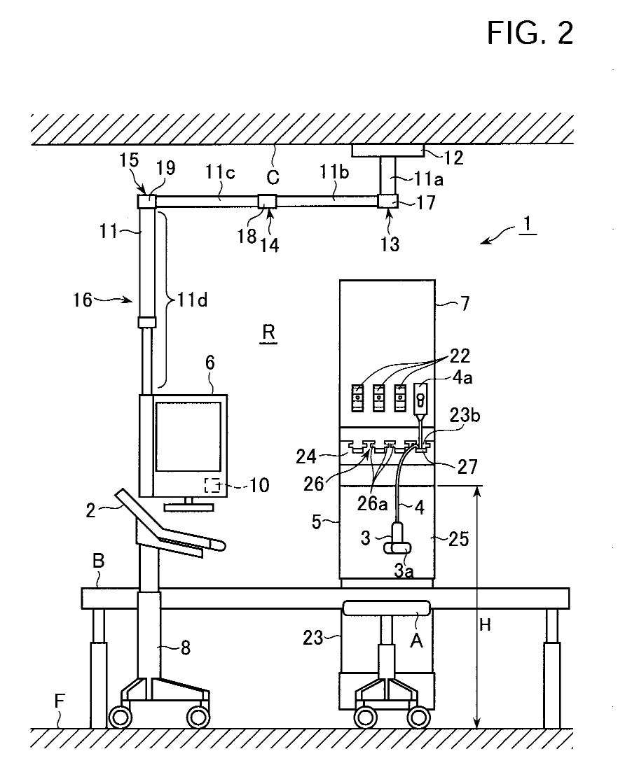

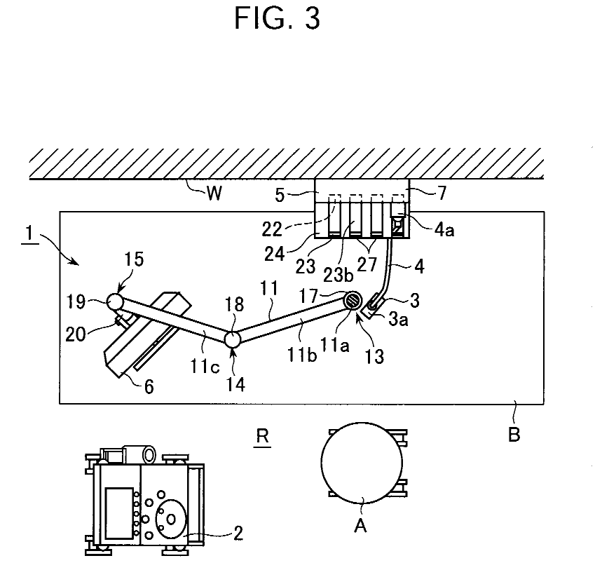

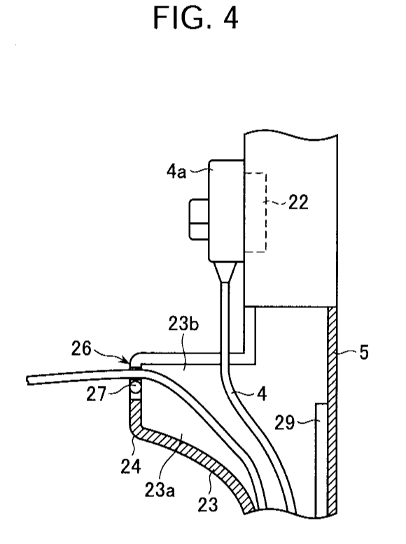

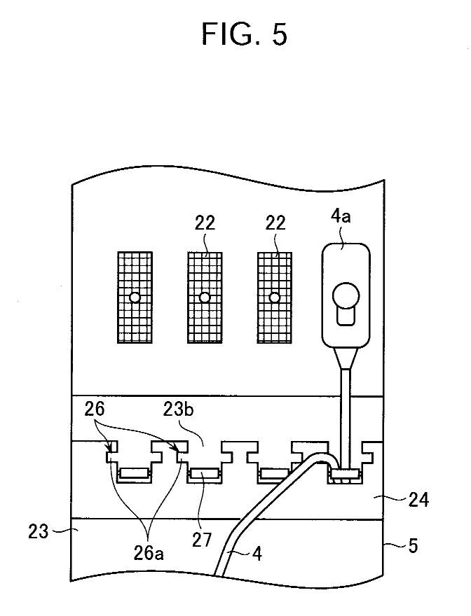

Firstly, a first embodiment will be described. FIG. 1 is a partially cutout side view showing an ultrasonic diagnostic apparatus according to the first embodiment of the invention, FIG. 2 is a front view of the ultrasonic diagnostic apparatus shown in FIG. 1, FIG. 3 is a plan view of the ultrasonic diagnostic apparatus shown in FIG. 1, FIG. 4 is a partially cutout enlarged side view showing the vicinity of an opening of the ultrasonic diagnostic apparatus shown in FIG. 1, and FIG. 5 is an enlarged front view showing the vicinity of an opening of the ultrasonic diagnostic apparatus shown in FIG. 1. In FIG. 3, a later-described first vertical arm 11a is illustrated in a cutout fashion.

An ultrasonic diagnostic apparatus 1 includes an operation device 2 by which an operator inputs an instruction, a probe 3 that transmits and receives an ultrasonic wave, a cable storing unit 5 that stores a cable 4 of the probe 3, an image display device 6 that displays an ultrasonic image, and a processing device 7 that controls to generate an ultrasonic image on the basis of an echo signal obtained through the drive of the probe 3 on the basis of the operator's instruction, and to display the obtained ultrasonic image onto the image display device 6.

The operation device 2 is separately provided from the image display device 6 and the processing device 7. It is placed onto a movable table 8 with casters, and is placed at the side where a chair A on which the operator sits on is placed with respect to a bed B on which a subject lies down. The operation device 2 has an operation-device-side wireless communication unit 9 for performing a wireless communication with the processing device 7. When the operator inputs the instruction by means of the operation device 2, the instruction signal is transmitted to the processing device 7 through the operation-device-side wireless communication unit 9.

The image display device 6 is separately provided from the processing device 7, and has an image-display-device-side wireless communication unit 10 for performing a wireless communication with the processing device 7. Data of the ultrasonic image is transmitted from the processing device 7 to the image display device 6, and this data is received by the image-display-device-side wireless communication unit 10.

The image display device 6 is mounted to a ceiling C of a room R, to which the ultrasonic diagnostic apparatus 1 is installed, through an arm 11. The arm 11 includes four arm members, which are a first vertical arm 11a, a first horizontal arm 11b, a second horizontal arm 11c, and a second vertical arm 11d. The image display device 6 is attached to the lower end of the second vertical arm 11d.

The first vertical arm 11a is provided to a base member 12 fixed to the ceiling C, and extends from the base member 12 in the vertical direction. The first horizontal arm 11b extends horizontally from the first vertical arm 11a, while the second horizontal arm 11c extends horizontally from the first horizontal arm 11b. The second vertical arm 11d extends vertically from the second horizontal arm 11c.

The arm 11 has, as a movable portion for changing the position of the image display device 6, a first movable portion 13, a second movable portion 14, a third movable portion 15, and a fourth movable portion 16. The first movable portion 13 is a first connection portion 17 where the first vertical arm 11a and the first horizontal arm 11b are connected to each other. The first horizontal arm 11b can be pivoted in the horizontal direction about the first connection portion 17. The second movable portion 14 is a second connection portion 18 where the first horizontal arm 11b and the second horizontal arm 11c are connected to each other. The first horizontal arm 11b and the second horizontal arm 11c can be pivoted in the horizontal direction about the second connection portion 18. The third movable portion 15 is a third connection portion 19 where the second horizontal arm 11c and the second vertical arm 11d are connected to each other. The second horizontal arm 11c and the second vertical arm 11d can be pivoted in the horizontal direction about the third connection portion 19. The fourth movable portion 16 is configured such that the second vertical arm 11d is extensible.

The image display device 6 is pivoted in the vertical direction at a connection portion 20 with the second vertical arm 11d, whereby the angle of a screen can be adjusted.

The processing device 7 is provided above the cable storing unit 5 so as to be integral, and attached to a wall W of the room R, to which the ultrasonic diagnostic apparatus 1 is installed, together with the cable storing unit 5. The processing device 7 has a processing-device-side wireless communication unit 21 for performing a wireless communication with the operation device 2 and the image display device 6. The data of the ultrasonic image generated at the processing device 7 is transmitted from the processing-device-side wireless communication unit 21 to the image display device 6. The processing device 7 also has a probe connector 22 for connecting a connector 4a of the cable 4.

The cable storing unit 5 is one example of the embodiment of the cable storing unit in the invention. The cable storing unit 5 has a cable storing casing 23 that is formed with a cable storing space 23a for storing the cable 4, which connects the probe body 3a of the probe 3 and the processing device 7, and that is attached to the wall W. The processing device 7 is integral with the cable storing casing 23. With the cable storing casing 23 attached to the wall, the cable storing casing 23 is approximately 150.0 centimeters (cm) high from a floor F, and the portion thereof having a height H approximately 100.0 cm (higher than the bed B) from the floor F has a thickness T of approximately 10.0 cm. In the invention, the thickness T is not limited to 10.0 cm. However, from the viewpoint of making the bed B as closer to the wall W as possible, the thickness T is desirably approximately 15.0 cm or less. With the cable storing casing 23 attached to the wall W, the portion having the height of between approximately 100.0 cm and approximately 130.0 cm from the floor F forms a projecting portion 24, and the thickness of the projecting portion 24 is approximately 20.0 cm.

A cover 25, which is slidable in the vertical direction, is mounted to the cable storing casing 23. When the cover 25 is slid in the downward direction, the inside of the portion of the cable storing casing 23 having the height between approximately 65.0 cm and approximately 100.0 cm from the floor F is exposed, whereby the operator can access to the cable storing space 23a. Thus, the operator easily carries out a maintenance operation. Specifically, the operator can remove the cable 4 of the probe 3 from a later-described movable pulley 28 or attach the cable 4 to the movable pulley 28.

The portion of the projecting portion 24 of the cable storing casing 23 having the height between approximately 120.0 cm and approximately 130.0 cm from the floor F forms an opening 23b. The probe connector 22 is positioned above the opening 23b, and the cable 4 of the probe 3 is stored in the cable storing space 23a from the opening 23b.

The cable 4 is stored in the cable storing space 23a as bent in a U-shape. Since the cable 4 is stored in this state, only one portion of the cable 4, i.e., the lower end of the U-shape (wound around the movable pulley 28) is bent, which prevents the cable 4 from being damaged.

A cable stopper 26 is provided to the opening 23b. The cable stopper 26 is a slit 26a that is narrower than the outer diameter of the cable 4. When the cable 4 is inserted into the slit 26a as elastically deformed, the cable 4 can be locked. Accordingly, this configuration can prevent the cable 4 from going out or prevent the cable 4 from being pulled into the cable storing space 23a against the intention of the operator.

A roller 27 that can be in contact with the lower surface of the cable 4 is provided to the opening 23b. By virtue of this roller 27, the cable 4 is smoothly inserted or removed into or from the opening 23b.

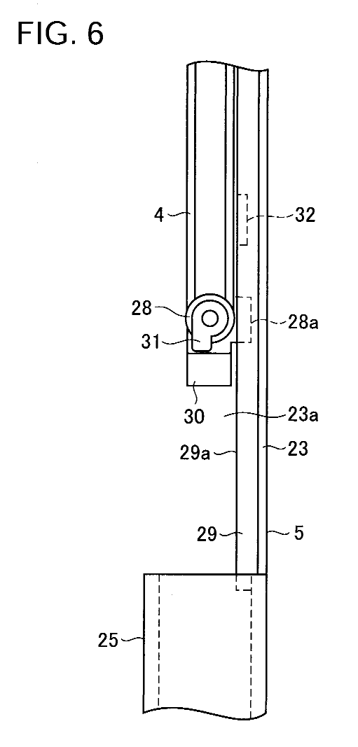

The internal configuration of the cable storing casing 23 will be explained with reference to FIG. 6. FIG. 6 is an enlarged side view showing the vicinity of the movable pulley 28 in which the cover 25 mounted to the cable storing casing 23 is slid downward to expose the inside of the portion of the cable storing casing 23 having the height between approximately 65.0 cm and approximately 100.0 cm from the floor F (not shown in FIG. 6).

The movable pulley 28 having a diameter of approximately 10.0 millimeters (mm) or more, for example, is provided at the lower end of the U-shaped cable 4. The movable pulley 28 can prevent the cable 4 from bending with a curvature smaller than the radius (e.g., 5.0 mm or more) of the movable pulley 28.

The movable pulley 28 is held by a rail 29, which is fixed in the vertical direction, at the back surface of the cable storing casing 23 in the cable storing space 23a, so as to be movable in the vertical direction through a holder 28a. The movable pulley 28 is biased downward by a weight 30. The weight 30 exerts a force for smoothly pulling the cable 4 into the cable storing space 23a.

A cable lock 31 is also provided to the movable pulley 28. The cable lock 31 is composed of a pivotable lever member. The cable lock 31 thus configured can prevent the cable 4 from being unlocked from the movable pulley 28, whereby stability and reliability can be enhanced.

A movable pulley lock 32 is mounted to the rail 29 at the position approximately 70.0 cm high from the floor F. The movable pulley lock 32 is a seesaw-like member that moves like a seesaw to change its posture. By virtue of this movable pulley lock 32, the movable pulley 28, from which the cable 4 is removed, can be locked to the position approximately 70.0 cm or more high from the floor F as described later, which provides an easy operation for attaching and detaching the cable 4 to and from the movable pulley 28.

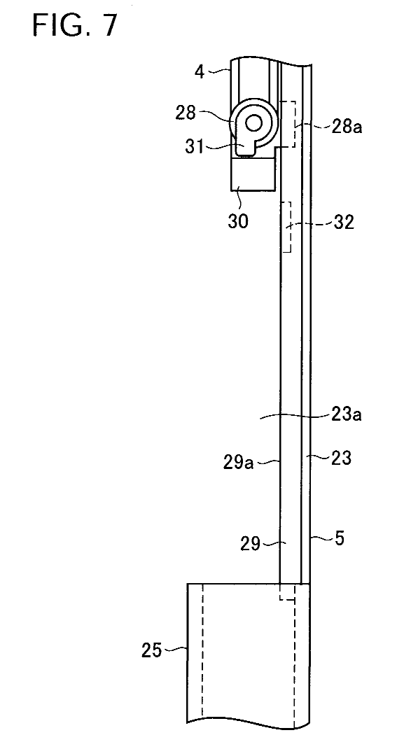

FIGS. 7 to 9 show a procedure for detaching the cable 4 from the movable pulley 28. In order to detach the cable 4 from the movable pulley 28, the cover 25 is firstly slid downward to expose the portion of the cable storing casing 23 having the height between approximately 65.0 cm and approximately 100.0 cm from the floor F (not shown in FIGS. 7 to 9), and with this state, the cable 4 is pulled out, and the movable pulley 28 is raised to the position above the movable pulley lock 32 as shown in FIG. 7.

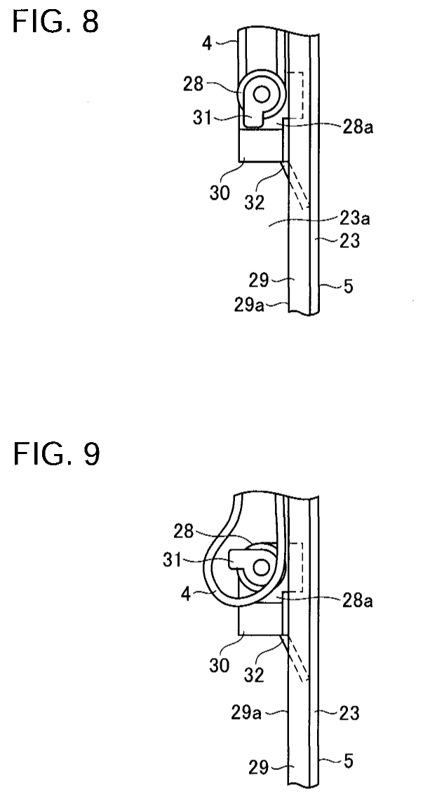

Next, as shown in FIG. 8, the movable pulley lock 32 is moved in a seesaw fashion to project the movable pulley lock 32 from the upper surface 29a of the rail 29, and then, the movable pulley 28 is lowered to put the same onto the movable pulley lock 32. Then, as shown in FIG. 9, the cable lock 31 is turned to move upward, whereby the cable 4 is detached from the movable pulley 28.

The procedure for attaching the cable 4 to the movable pulley 28 is the inverse order of the procedure for detaching the cable 4 from the movable pulley 28.

According to the ultrasonic diagnostic apparatus 1 of the present embodiment, the image display device 6 is separate from the operation device 2 and the processing device 7, and attached at the ceiling C through the arm 11. When the first horizontal arm 11b, the second horizontal arm 11c and the second vertical arm 11d are pivoted in the horizontal direction, and the second vertical arm 11d is extended or contracted to change its length, the operator can place the image display device 6 at the position where the operator can make a scan with a natural posture.

The operation device 2, which should be placed at the side where the operator is present across the bed B, and the processing device 7 are provided separately, and the processing device 7 is mounted to the wall W of the room R, whereby the processing device 7 can be arranged at the wall W side while the operation device 2 can be arranged at the side, which is opposite to the wall W side and where the operator is present, across the bed B. By virtue of this configuration, the space where the operator is present becomes wider than the conventional case because the processing device 7 is not placed in this space, so that the space of the room R can effectively be utilized.

The processing device 7 is integrally mounted above the cable storing casing 23 in which the thickness of the portion having a height of approximately 100.0 cm or less from the floor F of the room R is approximately 10.0 cm or less. Therefore, the gap between the bed B having the height of less than 100 cm and the wall W can be set to, for example, 11.0 cm, with the result that the bed B can be made as closer to the wall W as possible. Thus, the space where the operator is present becomes much wider than the conventional case, so that the space of the room R can effectively be utilized.

The cable storing casing 23 is formed with the opening 23a at the position having a height of approximately 120.0 cm or more from the floor F of the room R, whereby the cable 4 is inserted and removed at the position sufficiently higher than the patient lying down on the bed B in the room R. Accordingly, this configuration can prevent the patient from having an uncomfortable feeling by the cable 4.

The communication between the image display device 6 and the processing device 7 is wirelessly performed, so that the cable for connecting these devices can be eliminated. Therefore, this configuration can prevent the connection cable from hindering the movement of the image display device 6, and also can prevent the connection cable from becoming obstructive. Similarly, the communication between the operation device 2 and the processing device 7 is wirelessly performed, so that the cable for connecting these devices can be eliminated. Therefore, the operation device 2 can freely be moved by the movable table 8. Further, this configuration can prevent the connection cable from becoming obstructive.

The cable 4 can be stored in the cable storing casing 23. This configuration can prevent the cable 4 from becoming obstructive.

### Second Embodiment

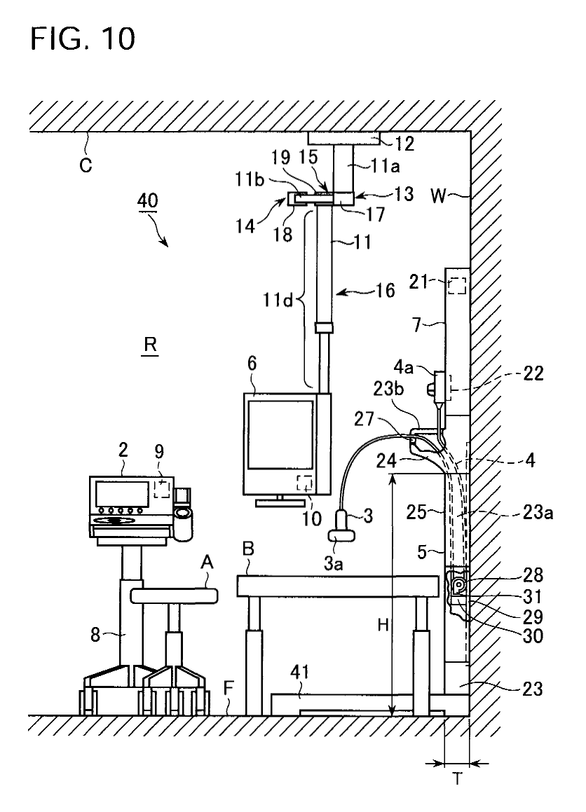

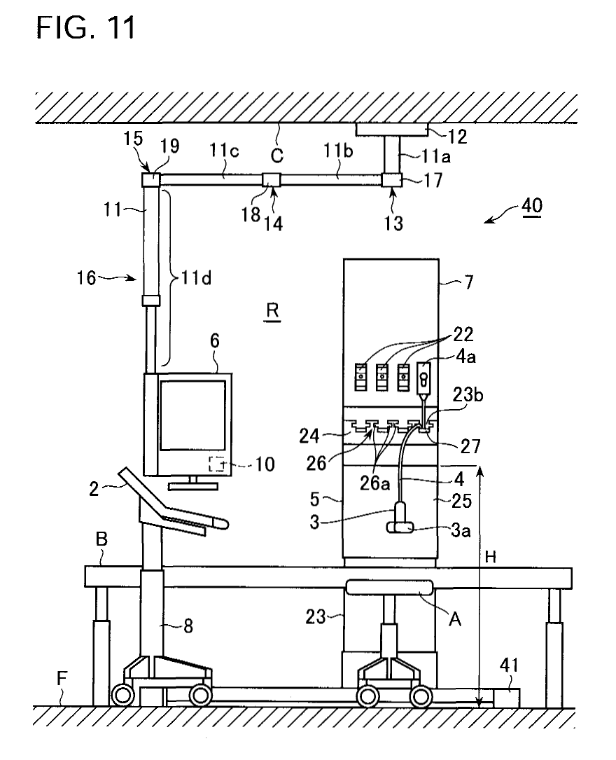

Next, the second embodiment according to the invention will be described with reference to FIGS. 10 and 11. FIG. 10 is a partially cutout side view showing an ultrasonic diagnostic apparatus according to the second embodiment, and FIG. 11 is a front view showing the ultrasonic diagnostic apparatus shown in FIG. 10.

In the ultrasonic diagnostic apparatus 40 in this embodiment, the processing device 7 and the cable storing unit 5 are arranged between the bed B and the wall W. Specifically, the cable storing unit 23 integrally formed with the processing device 7 is placed on the floor F. The cable storing casing 23 has a safety leg against overturning 41, and the cable storing casing 23 is stably placed on the floor F with the safety leg against overturning 41. The safety leg against overturning 41 is desirably fixed to the floor F with an anchor bolt (not shown).

In this embodiment, the cable storing casing 23 is approximately 150.0 cm high from the floor F, and the portion thereof having a height H approximately 100.0 cm from the floor F has a thickness T of approximately 10.0 cm. The thickness T is not limited to 10.0 cm, but the thickness T is desirably approximately 15.0 cm or less. The reason described in the first embodiment can be applied to the reason why the thickness T is desirably approximately 15.0 cm or less in this embodiment. The portion of the cable storing casing 23 having the height between approximately 100.0 cm and approximately 130.0 cm from the floor F forms a projecting portion 24, and the portion of the projecting portion 24 having the height between approximately 120.0 cm and approximately 130.0 cm is formed with the opening 23b.

According to the ultrasonic diagnostic apparatus 40 in the second embodiment, the processing device 5 is separately mounted from the operation device 2, and is placed to the vicinity of the wall W, whereby the space in the room R can effectively be utilized like the first embodiment. Further, the same advantage same as that in the first embodiment can be obtained.

### Third Embodiment

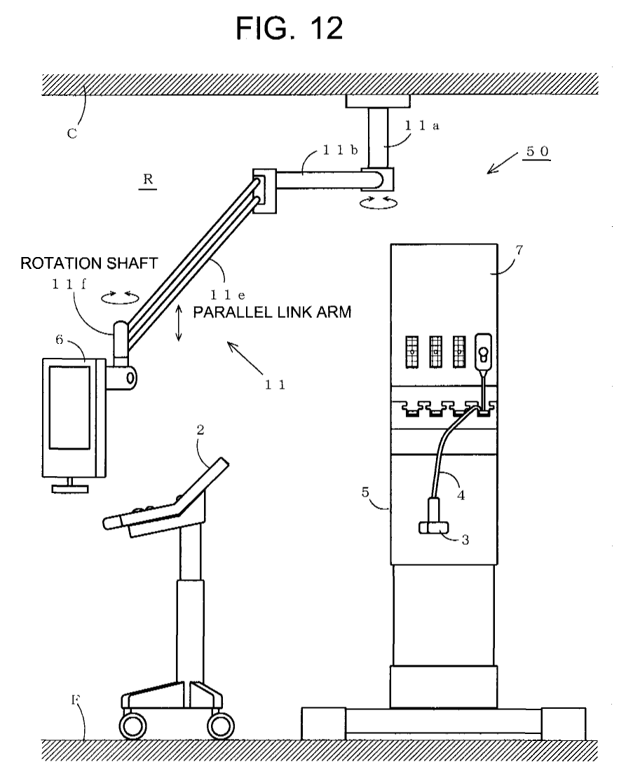

Next, the third embodiment according to the invention will be described with reference to FIG. 12. FIG. 12 is a front view showing an ultrasonic diagnostic apparatus according to the third embodiment of the invention.

In an ultrasonic diagnostic apparatus 50 in this embodiment, the arm 11 is composed of a first vertical arm 11a extending downward from the ceiling C, a first horizontal arm 11b horizontally extending from the lower end of the first vertical arm 11a so as to be pivotable, a parallel link arm 11e extending from the first horizontal arm 11b in the vertical direction so as to be deformed, and a rotation shaft 11f extending from the parallel link arm 11e and supporting the image display device 6 so as to be pivotable.

The parallel link arm 11e is a parallel link mechanism in which a horizontal side can be tilted with a vertical side kept to be vertical.

### Fourth Embodiment

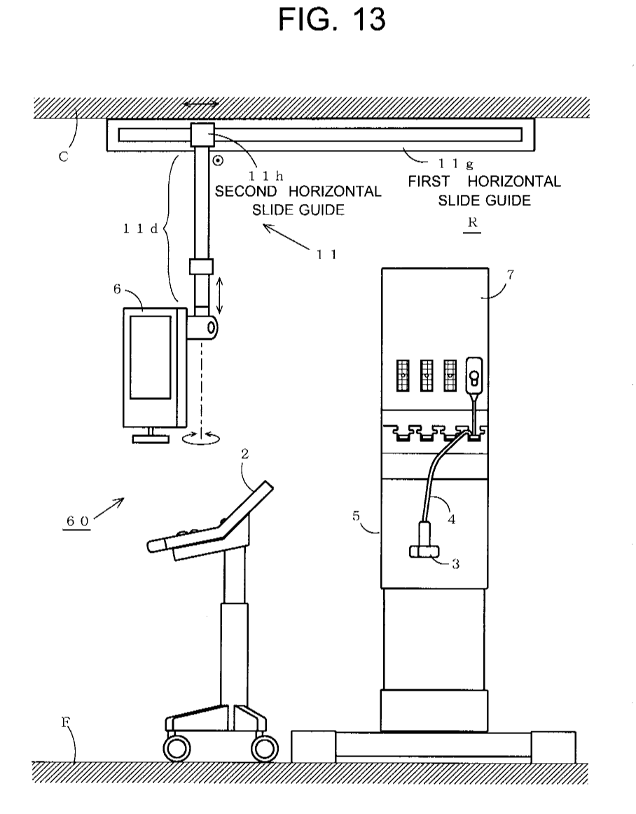

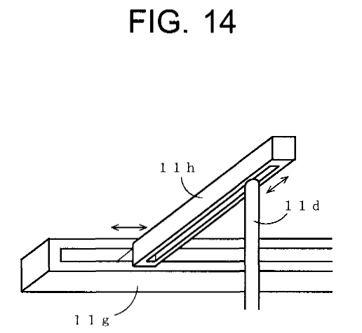

Next, the fourth embodiment according to the invention will be described with reference to FIGS. 13 and 14. FIG. 13 is a front view showing an ultrasonic diagnostic apparatus according to the fourth embodiment of the invention, and FIG. 14 is an upward view of a second horizontal slide guide according to the fourth embodiment of the invention.

In an ultrasonic diagnostic apparatus 60 in this embodiment, the arm 11 is composed of a first horizontal slide guide 11g attached to the ceiling C and extending laterally in the horizontal direction, a second horizontal slide guide 11h that is supported by the first horizontal slide guide 11g so as to be slidable in the lateral direction along the first horizontal slide guide 11g and that extends in the longitudinal direction, and a second vertical arm 11d that is supported along the second horizontal slide guide 11h so as to be slidable in the longitudinal direction and that supports the image display device 6 so as to be movable in the vertical direction and pivotable.

### Fifth Embodiment

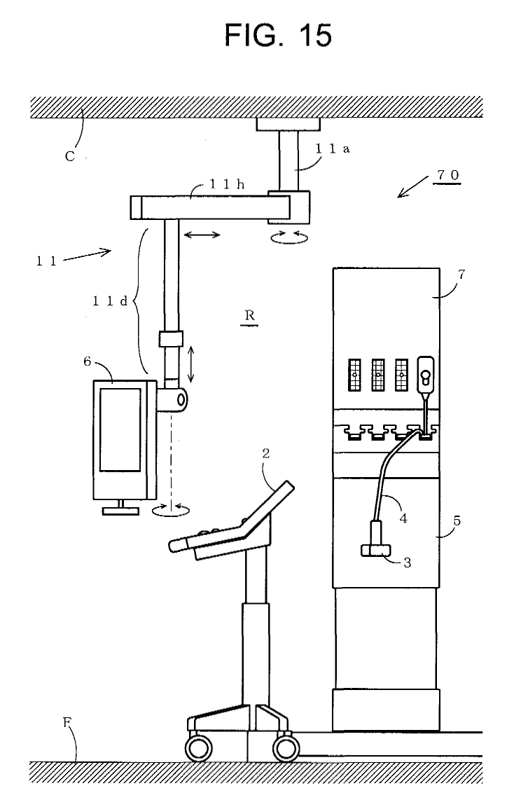

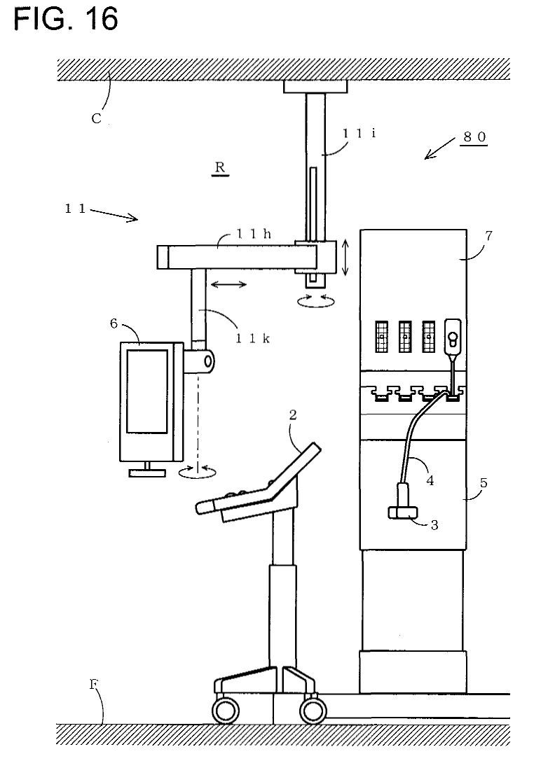

Next, the fifth embodiment according to the invention will be described with reference to FIG. 15. FIG. 15 is a front view showing an ultrasonic diagnostic apparatus according to the fifth embodiment of the invention.

In an ultrasonic diagnostic apparatus 70 in this embodiment, the arm 11 is composed of a first vertical arm 11a extending downward from the ceiling C, a second horizontal slide guide 11h horizontally extending from the lower end of the first vertical arm 11a so as to be pivotable, and a second vertical arm 11d that is supported along the second horizontal slide guide 11h so as to be slidable and that supports the image display device 6 so as to be movable in the vertical direction and pivotable.

### Sixth Embodiment

Next, the sixth embodiment according to the invention will be described with reference to FIG. 16. FIG. 16 is a front view showing an ultrasonic diagnostic apparatus according to the sixth embodiment of the invention.

In an ultrasonic diagnostic apparatus 80 in this embodiment, the arm 11 is composed of a rotatable first vertical slide guide 11i extending downward from the ceiling C, a second horizontal slide guide 11h that is supported by the first vertical slide guide 11i along the first vertical slide guide 11i so as to be slidable in the vertical direction and that horizontally extends, and a vertical arm 11k that is supported along the second horizontal slide guide 11h so as to be slidable and that supports the image display device 6 so as to be pivotable.

The invention has been described with reference to each embodiment. It is needless to say that various modifications are possible without departing from the scope of the invention. For example, although not especially shown, the arm 11 may be attached to the wall W, and the image display device 4 may be attached to the wall W through the arm 11. Although not shown, the arm 11 may be attached to the wall W through a rail or the like so as to be movable. Since the arm 11 is provided to be movable, the operator can place the image display device 6 at the position where the operator can make a scan with a natural posture. Further, when the operator does not use the apparatus, the operator can move the apparatus to the place in the room R where the apparatus does not become obstructive.

The cable storing unit in the invention may be formed as a part of the processing device 7. The processing device 7 may directly be placed onto the floor F. When the processing device 7 has a portion having a height of approximately 70. cm or less from the floor F, the thickness of this portion is desirably approximately 15.0 cm or less.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
an image display device configured to display an ultrasonic image; and
a processing device configured to generate the ultrasonic image based on an echo signal obtained through transmission of an ultrasonic wave, wherein said image display device is mounted separate from said processing device, and attached at a ceiling or a wall of a room, to which said ultrasonic diagnostic apparatus is installed, through an arm, said arm comprises a movable portion for changing a position of said image display device.

2. An ultrasonic diagnostic apparatus according to claim 1, wherein said arm comprises a plurality of arm members, said movable portion comprises a connection portion to which each of said plurality of arm members is pivotally connected.

3. An ultrasonic diagnostic apparatus according to claim 1, wherein said movable portion is configured such that said arm is extensible.

4. An ultrasonic diagnostic apparatus according to claim 2, wherein said movable portion is configured such that said arm is extensible.

5. An ultrasonic diagnostic apparatus according to claim 1, wherein said arm is mounted to the ceiling or the wall of the room, to which said ultrasonic diagnostic apparatus is installed, such that said arm is movable.

6. An ultrasonic diagnostic apparatus according to claim 2, wherein said arm is mounted to the ceiling or the wall of the room, to which said ultrasonic diagnostic apparatus is installed, such that said arm is movable.

7. An ultrasonic diagnostic apparatus according to claim 3, wherein said arm is mounted to the ceiling or the wall of the room, to which said ultrasonic diagnostic apparatus is installed, such that said arm is movable.

8. An ultrasonic diagnostic apparatus according to claim 4, wherein said arm is mounted to the ceiling or the wall of the room, to which said ultrasonic diagnostic apparatus is installed, such that said arm is movable.

9. An ultrasonic diagnostic apparatus according to claim 1, wherein said image display device and said processing device each comprise a respective wireless communication unit configured for mutual wireless communication.

10. An ultrasonic diagnostic apparatus according to claim 1, further comprising an operation device by which an operator inputs an instruction, wherein said processing device is provided separate from said operation device, and is mounted to a wall of the room to which said ultrasonic diagnostic apparatus is installed, or in a vicinity of the wall of the room.

11. An ultrasonic diagnostic apparatus according to claim 10, wherein said operation device is placed onto a movable table with casters.

12. An ultrasonic diagnostic apparatus according to claim 10, wherein said image display device and said processing device each comprise a respective wireless communication unit configured for mutual wireless communication.

13. An ultrasonic diagnostic apparatus according to claim 1, wherein a probe configured to transmit and receive the ultrasonic wave is connected to said processing device, said ultrasonic diagnostic apparatus further comprising a cable storing unit configured to store a cable for connecting a probe body of said probe with said processing device.

14. An ultrasonic diagnostic apparatus according to claim 2, wherein a probe configured to transmit and receive the ultrasonic wave is connected to said processing device, said ultrasonic diagnostic apparatus further comprising a cable storing unit configured to store a cable for connecting a probe body of said probe with said processing device.

15. An ultrasonic diagnostic apparatus according to claim 3, wherein a probe configured to transmit and receive the ultrasonic wave is connected to said processing device, said ultrasonic diagnostic apparatus further comprising a cable storing unit configured to store a cable for connecting a probe body of said probe with said processing device.

16. An ultrasonic diagnostic apparatus according to claim 5, wherein a probe configured to transmit and receive the ultrasonic wave is connected to said processing device, said ultrasonic diagnostic apparatus further comprising a cable storing unit configured to store a cable for connecting a probe body of said probe with said processing device.

17. An ultrasonic diagnostic apparatus according to claim 13, wherein said cable storing unit comprises a cable storing casing having a storing space for said cable formed therein.

18. An ultrasonic diagnostic apparatus according to claim 17, wherein said cable storing casing is formed with an opening at a position having a height of 120 cm or more from a floor of the room, and said cable is stored into said cable storing space from said opening.

19. An ultrasonic diagnostic apparatus according to claim 17, wherein said processing device is integrally mounted above said cable storing casing in which a thickness of a portion having a height of 70 cm or less from a floor of the room is 15 cm or less.

20. An ultrasonic diagnostic apparatus according to claim 1, wherein said processing device comprises a portion having a height of 70 cm or less from a floor of the room, a thickness of said portion is 15 cm or less.
